SEQUENCE LISTING (2 sequences)

SEQ ID NO: 1 - protein, 16 aa, homo sapiens
EARPALLTSRLRFIPK

SEQ ID NO: 2 - protein, 1132 aa, homo sapiens
MPRAPRCRAVRSLLRSHYREVLPLATFVRRLGPQGWRLVQRGDPAAFRALVAQCLVCVPWDARPPPAAPSFRQVSCLKELVARVLQRLCERGAKNVLAFGFALLDGARGGPPEAFTTSVRSYLPNTVTDALRGSGAWGLLLRRVGDDVLVHLLARCALFVLVAPSCAYQVCGPPLYQLGAATQARPPPHASGPRRRLGCERAWNHSVREAGVPLGLPAPGARRRGGSASRSLPLPKRPRRGAAPEPERTPVGQGSWAHPGRTRGPSDRGFCVVSPARPAEEATSLEGALSGTRHSHPSVGRQHHAGPPSTSRPPRPWDTPCPPVYAETKHFLYSSGDKEQLRPSFLLSSLRPSLTGARRLVETIFLGSRPWMPGTPRRLPRLPQRYWQMRPLFLELLGNHAQCPYGVLLKTHCPLRAAVTPAAGVCAREKPQGSVAAPEEEDTDPRRLVQLLRQHSSPWQVYGFVRACLRRLVPPGLWGSRHNERRFLRNTKKFISLGKHAKLSLQELTWKMSVRDCAWLRRSPGVGCVPAAEHRLREEILAKFLHWLMSVYVVELLRSFFYVTETTFQKNRLFFYRKSVWSKLQSIGIRQHLKRVQLRELSEAEVRQHREARPALLTSRLRFIPKPDGLRPIVNMDYVVGARTFRREKRAERLTSRVKALFSVLNYERARRPGLLGASVLGLDDIHRAWRTFVLRVRAQDPPPELYFVKVDVTGAYDTIPQDRLTEVIASIIKPQNTYCVRRYAVVQKAAHGHVRKAFKSHVSTLTDLQPYMRQFVAHLQETSPLRDAVVIEQSSSLNEASSGLFDVFLRFMCHHAVRIRGKSYVQCQGIPQGSILSTLLCSLCYGDMENKLFAGIRRDGLLLRLVDDFLLVTPHLTHAKTFLRTLVRGVPEYGCVVNLRKTVVNFPVEDEALGGTAFVQMPAHGLFPWCGLLLDTRTLEVQSDYSSYARTSIRASLTFNRGFKAGRNMRRKLFGVLRLKCHSLFLDLQVNSLQTVCTNIYKILLLQAYRFHACVLQLPFHQQVWKNPTFFLRVISDTASLCYSILKAKNAGMSLGAKGAAGPLPSEAVQWLCHQAFLLKLTRHRVTYVPLLGSLRTAQTQLSRKLPGTTLTALEAAANPALPSDFKTILD

(19) United States
(12) Patent Application Publication
(10) Pub. No.: US 2017/0128557 A1
(43) Pub. Date: May 11, 2017

(54) Dendritic Cell Therapeutic Agent and Immunotherapeutic Agent Comprising Peptide Derived from Telomerase, and Therapeutic Methods Using the Same

(71) Applicants: Sang Jae Kim, Seoul (KR); GemVax & KAEL Co., Ltd., Daejeon (KR); Korea Stem Cell Bank, Gyeonggi-do (KR)

(72) Inventors: Sang Jae KIM, Seoul (KR); Hiroyuki Abe, Tokyo (JP); Hwa In Jang, Kangwon-do (KR); Jung Soon Ha, Gyeonggi-do (KR)

(73) Assignee: GemVax & KAEL Co., Ltd., Daejeon (KR)

(21) Appl. No.: 15/346,870

(22) Filed: Nov. 9, 2016

(30) Foreign Application Priority Data
Nov. 9, 2015 (KR) 10-2015-0156996

(51) Int. Cl. A61K 39/00; A61K 45/06; A61K 35/17

(52) U.S. Cl. CPC A61K 39/0011; A61K 35/17; A61K 45/06; A61K 2039/5154; A61K 2039/545

(57) ABSTRACT

Provided is a dendritic cell therapeutic agent, and more particularly, a composition which include dendritic cells activated by peptides including a telomerase-derived peptide, the composition administered to treat an individual having disease and disorder symptoms which require target-specific treatments. Also, provided is a therapeutic method effective on diseases requiring target-specific immunotherapy. In the method, co-administration of the dendritic cell therapeutic agent and an immunotherapeutic agent including the telomerase-derived peptide results in decreased factors causing one of the disease and disorder symptoms requiring the target-specific treatment, such as cancer, in a tumor disease treatment.

### CROSS-REFERENCE TO RELATED APPLICATION

This application claims priority to and the benefit of Korean Patent Application No. 10-2015-0156996, filed on Dec. 9, 2015, the disclosure of which is incorporated herein by reference in its entirety.

### BACKGROUND

1. Field of the Invention

The present invention relates to an immune response-activating composition, which comprises a dendritic cell therapeutic agent including dendritic cells stimulated by a telomerase-derived peptide inducing an immune response and peptides including the same and/or an immunotherapeutic agent including a telomerase-derived peptide, and a method for activating an immune response by administering the dendritic cell therapeutic agent alone and/or in combination with the immunotherapeutic agent. More particularly, the present invention relates to a dendritic cell therapeutic agent comprising dendritic cells activated by peptides including a telomerase-derived peptide and a composition comprising the same, which is used alone or in combination with an immunotherapeutic agent including a telomerase-derived peptide to be effective in treating inflammations or diseases including cancer due to increased antigen-specific immune response, and to an immunotherapeutic agent administered in combination with the cell therapeutic agent.

2. Discussion of Related Art

A cell therapeutic agent is a medicine used to treat, diagnose, and prevent a chain of behaviors such as in vitro proliferating and selecting live autologous, allogenic, or xenogenic cells to restore functions of cells and tissue, or changing biological characteristics of cells using another method.

A dendritic cell immunotherapeutic agent among the cell therapeutic agents is prepared from very specialized immune cells such as dendritic cells derived from a subject to which the immunotherapeutic agent is administered activated by pulsing peptides thereonto, and administered into the body of a target requiring a therapeutic agent. The administered dendritic cells present antigens of corresponding peptides to T cells, and the T cells (cytotoxic T lymphocytes: CTLs) activated thereby specifically attack cells presenting the corresponding peptides, and therefore a disease may be treated without damaging normal cells in the body. Immature DCs (Dendritic cells) cannot stimulate T cells but have an excellent antigen-capturing ability. Immature DCs are stimulated to differentiate into mature dendritic cells by capturing antigens and other stimulating substances. Mature DCs presenting antigens strongly express CD80, CD83, CD86, and MHC class I and class II, migrate to a paracortical region in which T cells of a draining lymph node are abundant to present an antigen to the T cells, induce an antigen-specific T cell cytotoxic response (cytotoxic T lymphocytes, CTLs), and induce helper T cells, resulting in an anticancer effect.

However, dendritic cells needed for preparing the dendritic cell immunotherapeutic agent may not be directly separated from the body. For this reason, the dendritic cells are obtained by separating mononuclear cells from the bone marrow or blood taken from a subject to which the immunotherapeutic agent is administered and differentiating the mononuclear cells into the dendritic cells.

As a conventionally known method for collecting mononuclear cells used to prepare a dendritic cell immunotherapeutic agent, apheresis is known. The apheresis refers to collection of only the required ingredients by passing whole blood extracted from a donor or recipient through an instrument. According to apheresis, a method for separating leukocytes from blood using an apheresis instrument is known. However, apheresis incurs expense for operation, and high-level technical skills for operating the instrument are needed. Also, in apheresis, a mixture is extracted including mononuclear cells and other ingredients (leukocytes, erythrocytes or thrombocytes). For this reason, after performing apheresis, a process of separating mononuclear cells is generally performed to remove ingredients except the mononuclear cells, such as erythrocytes or thrombocytes.

To apply the dendritic cell immunotherapeutic agent to clinical trials, approximately 1×10⁷ cells are preferably used for a single administration. To obtain such a number of the cells, usually, apheresis is performed on the same target approximately 8 times, with an interval between each thereof. Also, since the blood contains a lower ratio of the mononuclear cells, for apheresis performed to obtain a sufficient amount of the mononuclear cells to prepare the dendritic cell immunotherapeutic agent, circulation of the blood in an apheresis instrument is needed to sufficiently extract leukocyte ingredients, which is also physically demanding and time consuming for a patient. For this reason, when condition of a patient is dramatically changed during an apheresis procedure, the apheresis procedure may be stopped, and the dendritic cell immunotherapy itself may be abandoned. Also, through apheresis, while it is thought that the mononuclear cell ingredients may be collected at an amount that is sufficient for preparing the dendritic cell immunotherapeutic agent generally 5 to 8 times, an actual amount of the dendritic cell immunotherapeutic agent obtained changes according to the blood condition of a patient. Another disadvantage of the apheresis is that the collected mononuclear cells have to be stored in a frozen state, and therefore such frozen cells have to be thawed before administration.

A method for preparing dendritic cells for an immunotherapeutic agent without using apheresis includes collecting and culturing mononuclear cells obtained from the peripheral blood of a patient, activating immature dendritic cells differentiated from the mononuclear cells by pulsing peptides thereonto, and activating mature dendritic cells obtained in the previous procedure by pulsing peptides onto the matured dendritic cells again. The method for preparing dendritic cells for an immunotherapeutic agent without using apheresis may reduce pain for a patient and time consumption, which are disadvantages of apheresis. Also, in this method, one vaccine can be prepared with approximately 25 to 30 ml of peripheral blood only. In addition, fresh vaccines may be obtained without freezing or thawing dendritic cells.

Cell-mediated immunity is accomplished by cells serving to killing infected or transformed cells (cancer cells, etc.), for example, CTLs and natural killer cells (NK cells).

CTLs are cells for antigen-specific cell-mediated immunity. That is, they recognize specific antigens to kill cells presented by the antigens through apoptosis. Such immunity is antigen-specific and thus is classified as adaptive immunity. Activated CTLs serve to attack and kill target cells by secreting a substance such as perforin or granzyme.

NK cells are different from CTLs in that the NK cells do not anti-specifically recognize cells having problems (virus-infected or transformed cells such as cancer cells). Such immunity is not antigen-specific and thus is classified as innate immunity. NK cells serve to attack and kill cells presenting peptides that are originally included in an individual at an abnormally low level.

Most of the various diseases including inflammation or cancer triggered by cellular infection and degeneration cells are treated through extensive treatments including various chemotherapies and radiation therapies. Many side effects may occur due to much loss and death of normal cells, which have not been infected or degenerated in the above-described process. Therefore, there is a need for a therapeutic agent specifically attacking and removing defective cells due to inflammation or degeneration without damaging normal cells.

A peptide according to the present invention (hereinafter, referred to PEP1) is known as a peptide composed of 16 major amino acids residing in a catalytic domain of telomerase to have anti-inflammatory and antioxidative effects. As clinical trials have found that a composition for immunotherapy comprising dendritic cells activated by pulsing a peptide group including PEP1 is effective for various diseases including inflammation or cancer induced by cellular infection and degeneration, the composition is expected to show an effect of target-specific immunity for various diseases including inflammation or cancer. The present invention also shows that an immunotherapeutic agent comprising PEP1 is administered in combination with a dendritic cell therapeutic agent activated by antigen peptides including the PEP1, resulting in an immune-boosting effect. Moreover, the present invention shows that the immunotherapeutic agent comprising the PEP1 is used in combination with the dendritic cell therapeutic agent activated by the antigen peptides including the PEP1 and an NK cell therapeutic agent, thereby obtaining an increased immune effect.

The present invention provides an immune response-activating composition for generating an immune response to inflammation or cancer, the composition comprising a dendritic cell therapeutic agent activated by peptides including a peptide derived from a reverse transcriptase of telomerase and an immunotherapeutic agent including the telomerase-derived peptide. Specifically, the present invention provides a composition for activating an immune response induced by administering dendritic cells activated by peptides including a human reverse transcriptase of human telomerase (hTERT)-derived amino acid peptide alone or in combination with an immunotherapeutic agent including the telomerase-derived peptide.

### PRIOR ART DOCUMENTS

### Patent Documents

(Patent Document 1) WO 2013-118899 A1

(Patent Document 2) WO 2013-100500 A1

(Patent Document 3) JP 5577472 B2

### SUMMARY OF THE INVENTION

Under such a background, the inventors attempted to develop dendritic cells activated by a peptide, having an excellent effect and almost no side effect, and a dendritic cell therapeutic agent, and thus completed the present invention.

The present invention is directed to providing a composition for activating an immune response induced by administering a dendritic cell therapeutic agent, which is effective, has no side effect, and is activated by a telomerase-derived peptide alone or in combination with an immunotherapeutic agent including the telomerase-derived peptide, and a method for treating diseases including inflammation or cancer using the composition.

According to an aspect of the present invention, an immune response-activating composition is provided, for generating an immune response to infected or degenerated cells including dendritic cells activated by one or more peptides selected from the group consisting of a peptide having an amino acid sequence of SEQ ID NO: 1, a peptide having at least 80% sequence homology with the amino acid sequence and a fragment thereof.

In the composition according to the present invention, the fragment may be composed of three or more amino acids.

In the composition according to the present invention, the dendritic cells may be further activated by one or more antigen-derived peptides selected from the group consisting of WT1, MUC-1, CA125, MAGE-A3, CEA, NY-ESO1, Survinin and Her2 in addition to PEP1.

The composition according to the present invention may treat inflammation or cancer by generating an immune response to infected or degenerated cells.

In the composition according to the present invention, the cancer may be at least one selected from pancreatic cancer, lung cancer, breast cancer, prostatic cancer, liver cancer and renal cancer.

In the composition according to the present invention, the dendritic cells may be derived from mononuclear cells cultured after being selected from the peripheral blood of an individual subjected to the administration of the composition.

In the composition according to the present invention, the composition may be administered in combination with an immunotherapeutic agent including a peptide of SEQ ID NO: 1 or a fragment thereof.

In the composition according to the present invention, the composition may be administered in combination with an NK cell therapeutic agent.

In the composition according to the present invention, the composition may be administered in combination with one or more drugs for anticancer chemotherapy or target anticancer agent.

In the composition according to the present invention, the composition may be used in combination with radiation therapy

According to another aspect of the present invention, a method for activating an immune response to generate an immune response to inflammation or cancer is provided, the method comprising administering an immune response-activating composition, which comprises a pharmaceutically effective amount of the dendritic cells to an individual with disease or disorder symptoms requiring a target-specific treatment.

In the method for activating an immune response according to the present invention, the composition may be administered by intradermal injection around a lymph node every two weeks.

In the method for activating an immune response according to the present invention, the composition may be administered in combination with an immunotherapeutic agent comprising a peptide of SEQ ID NO: 1 or a fragment thereof.

According to another aspect of the present invention, an immune response-activating kit for generating an immune response to inflammation or cancer, the kit comprising the immune response-activating composition comprising dendritic cells.

In the kit according to the present invention, the kit further comprises an immunotherapeutic agent comprising a peptide of SEQ ID NO: 1 or a fragment thereof.

In the kit according to the present invention, the kit may comprise an instruction directing administration of the immune response-activating composition and the immunotherapeutic agent of SEQ ID NO: 1 according to the present invention, which comprise dendritic cells as a pharmaceutically active ingredient, through intradermal injection around a lymph node every two weeks.

### BRIEF DESCRIPTION OF THE DRAWINGS

The above and other objects, features and advantages of the present invention will become more apparent to those of ordinary skill in the art by describing in detail exemplary embodiments thereof with reference to the accompanying drawings, in which:

FIG. 1A shows a result of analyzing PEP1 antigen endocytosis of immature dendritic cells according to the present invention through flow cytometry, and FIG. 1B shows a result of observing the PEP1 antigen uptake of the immature dendritic cells according to the present invention through confocal microscopy;

FIG. 2 shows a result of analyzing the induction of antigen-specific T cell proliferation of PEP1-activated dendritic cells according to the present invention;

FIG. 3 shows a result of analyzing IFN-γ secretion from PEP1-specific T cells according to the present invention;

FIG. 4 shows images illustrating a result of the ELISPOT assay on T cells of the peripheral blood of a 74 year-old female patient with stomach cancer (Patient 1) before dendritic cell immunotherapy according to the present invention, and a table showing the quantified result thereof;

FIG. 5 shows images illustrating a result of the ELISPOT assay on T cells in the peripheral blood of the 74 year-old female patient with stomach cancer (Patient 1) after the dendritic cell immunotherapy according to the present invention, and a table showing the quantified result thereof;

FIG. 6 shows images illustrating a result of the ELISPOT assay on T cells in peripheral blood of a 77 year-old male patient with lung cancer (Patient 2) before dendritic cell immunotherapy according to the present invention, and a table showing the quantified result thereof;

FIG. 7 shows images illustrating a result of the ELISPOT assay on T cells in peripheral blood of the 77 year-old male patient with lung cancer (Patient 2) after the dendritic cell immunotherapy according to the present invention, and a table showing the quantified result thereof;

FIG. 8 shows images illustrating a result of the ELISPOT assay on T cells in the peripheral blood of a 72 year-old male patient with pancreatic cancer (Patient 3) before dendritic cell immunotherapy according to the present invention, and a table showing the quantified result thereof;

FIG. 9 shows images illustrating a result of the ELISPOT assay on T cells in the peripheral blood of the 72 year-old male patient with pancreatic cancer (Patient 3) after the dendritic cell immunotherapy according to the present invention, and a table showing the quantified result thereof;

FIG. 10 shows images illustrating a result of the ELISPOT assay on T cells in the peripheral blood of a 66 year-old female patient with breast cancer (Patient 4) before dendritic cell immunotherapy according to the present invention, and a table showing the quantified result thereof; and

FIG. 11 shows images illustrating a result of the ELISPOT assay on T cells in the peripheral blood of the 66 year-old female patient with breast cancer (Patient 4) after the dendritic cell immunotherapy according to the present invention, and a table showing the quantified result thereof.

### DETAILED DESCRIPTION OF EXEMPLARY EMBODIMENTS

The present invention may be modified in various forms and have many examples, and thus will be described in detail based on the examples below. However, these examples are not provided to limit the present invention to specific embodiments, and it should be understood that the present invention can have various examples and applications as described in the claims, and comprises all modifications, equivalents and alternatives within the spirit and technical scope of the present invention. To explain the present invention, if it is determined that a detailed description of related art may obscure the gist of the present invention, the detailed description thereof will be omitted.

In one aspect of the present invention, a peptide of SEQ ID NO: 1, a fragment thereof or a peptide having at least 80% sequence homology with the peptide sequence includes a peptide derived from telomerase, specifically, Homo sapiens telomerase.

The peptide disclosed in the specification may include a peptide having 80% or higher, 85% or higher, 90% or higher, 95% or higher, 96% or higher, 97% or higher, 98% or higher, or 99% or higher sequence homology. Also, the peptide disclosed in the specification may include a peptide having at least one, two, three, four, five, six, or seven different amino acids from the peptide of SEQ ID NO: 1 or a fragment thereof.

In one aspect of the present invention, amino acid change is one of the properties that change physicochemical characteristics of the peptide. For example, amino acids may be changed to enhance thermal stability, change substrate specificity, and shift an optimal pH of the peptide.

Also, the peptide of SEQ ID NO: 1, fragment thereof or a peptide having at least 80% sequence homology with the peptide sequence according to one aspect of the present invention has low cytotoxicity and high in vivo stability. In the present invention, the SEQ ID NO: 1 is a telomerase-derived peptide composed of 16 amino acids as listed below.

The peptide set forth in SEQ ID NO: 1 is shown in Table 1 below. The “name” in Table 1 below is given to distinguish one peptide to another. In one aspect of the present invention, the peptide set forth in SEQ ID NO: 1 represents the whole peptide of Homo sapiens telomerase. In another aspect of the present invention, the peptide of SEQ ID NO: 1, a fragment thereof or a peptide having at least 80% sequence homology with the peptide sequence includes a “synthetic peptide” synthesized from a peptide present at a corresponding location of the peptides included in the telomerase. SEQ. ID. NO: 2 denotes the full-length amino acid sequence of the telomerase.

TABLE 1

| SEQ ID | | Position on | | |
|---|---|---|---|---|
| NO: | Name | telomerase | Sequence | Length |
| 1 | PEP1 | [611-626] | EARPALLTSRLRFIPK | 16 aa |
| 2 | | [1-1132] | MPRAPRCRAVRSLLRSHYREVLPLA | 1132 aa |
| | | | TFVRRLGPQGWRLVQRGDPAAFRA | |
| | | | LVAQCLVCVPWDARPPPAAPSFRQ | |
| | | | VSCLKELVARVLQRLCERGAKNVL | |
| | | | AFGFALLDGARGGPPEAFTTSVRSY | |
| | | | LPNTVTDALRGSGAWGLLLRRVGD | |
| | | | DVLVHLLARCALFVLVAPSCAYQV | |
| | | | CGPPLYQLGAATQARPPPHASGPRR | |
| | | | RLGCERAWNHSVREAGVPLGLPAP | |
| | | | GARRRGGSASRSLPLPKRPRR | |
| | | | GAAPEPERTPVGQGSWAHPGRTRG | |
| | | | PSDRGFCVVSPARPAEEATSLEGAL | |
| | | | SGTRHSHPSVGRQHHAGPPSTSRPP | |
| | | | RPWDTPCPPVYAETKHFLYSSGDKE | |
| | | | QLRPSFLLSSLRPSLTGARRLVETIFL | |
| | | | GSRPWMPGTPRRLPRLPQRYWQMR | |
| | | | PLFLELLGNHAQCPYGVLLKTHCPL | |
| | | | RAAVTPAAGVCAREKPQGSVAAPE | |
| | | | EEDTDPRRLVQLLRQHSSPWQVYG | |
| | | | FVRACLRRLVPPGLWGSRHNERRFL | |
| | | | RNTKKFISLGKHAKLSLQELTWKMS | |
| | | | VRDCAWLRRSPGVGCVPAAEHRLR | |
| | | | EEILAKFLHWLMSVYVVELLRSFFY | |
| | | | VTETTFQKNRLFFYRKSVWSKLQSI | |
| | | | GIRQHLKRVQLRELSEAEVRQHREA | |
| | | | RPALLTSRLRFIPKPDGLRPIVNMDY | |
| | | | VVGARTFRREKRAERLTSRVKALFS | |
| | | | VLNYERARRPGLLGASVLGLDDIHR | |
| | | | AWRTFVLRVRAQDPPPELYFVKVD | |
| | | | VTGAYDTIPQDRLTEVIASIIKPQNT | |
| | | | YCVRRYAVVQKAAHGHVRKAFKS | |
| | | | HVSTLTDLQPYMRQFVAHLQETSPL | |
| | | | RDAVVIEQSSSLNEASSGLFDVFLRF | |
| | | | MCHHAVRIRGKSYVQCQGIPQGSIL | |
| | | | STLLCSLCYGDMENKLFAGIRRDGL | |
| | | | LLRLVDDFLLVTPHLTHAKTFLRTL | |
| | | | VRGVPEYGCVVNLRKTVVNFPVED | |
| | | | EALGGTAFVQMPAHGLFPWCGLLL | |
| | | | DTRTLEVQSDYSSYARTSIRASLTFN | |
| | | | RGFKAGRNMRRKLFGVLRLKCHSL | |
| | | | FLDLQVNSLQTVCTNIYKILLLQAY | |
| | | | RFHACVLQLPFHQQVWKNPTFFLR | |
| | | | VISDTASLCYSILKAKNAGMSLGAK | |
| | | | GAAGPLPSEAVQWLCHQAFLLKLT | |
| | | | RHRVTYVPLLGSLRTAQTQLSRKLP | |
| | | | GT TLTALEAAANPALPSDFKTILD | |

In one aspect of the present invention, an immune response-activating composition is provided, comprising dendritic cells activated by peptides including a peptide having an amino acid sequence of SEQ ID NO: 1, a peptide having at least 80% sequence homology with the amino acid or a fragment thereof as an active ingredient.

The immune response-activating composition comprising dendritic cells activated by peptides according to one aspect of the present invention may be administered in combination with an immunotherapeutic agent comprising an amino acid sequence of SEQ ID NO: 1 or a fragment thereof.

The immune response-activating composition according to an aspect of the present invention may be applied to all animals including a human, a dog, a chicken, a pig, a cow, a sheep, a guinea pig, and a monkey.

The immune response-activating composition according to an aspect of the present invention may be transdermally, intravenously, intramuscularly, intraperitoneally, intraosseously, intrathecally or subcutaneously administered.

The immune response-activating composition according to an aspect of the present invention may comprise, as needed, additives such as diluents, excipients, lubricants, binders, disintegrants, buffers, dispersants, surfactants, coloring agents, aromatics, or sweeteners. The immune response-activating composition according to an aspect of the present invention may be prepared by a conventional method in the art.

Dosages of the immunotherapeutic agent comprising the amino acid sequence of SEQ ID NO: 1 or a fragment thereof administered in combination with the immune response-activating composition according to an aspect of the present invention may vary according to a patient's age, sex, weight, pathological state and severity, an administration route, or a prescriber's judgment. The dosage based on such factors may be determined at the level of one of ordinary skill in the art, and a daily dose of the immunotherapeutic agent may be, for example, 0.1 μg/kg/day to 100 g/kg/day, specifically, 10 μg/kg/day to 10 g/kg/day, more specifically, 100 μg/kg/day to 1 g/kg/day, and further more specifically, 500 μg/kg/day to 100 mg/kg/day, and may be suitably adjusted when there is a difference in effects according to doses. The pharmaceutical composition according to one aspect of the present invention may be administered one to three times a day, but the present invention is not limited thereto.

The terms used in the specification are intended to be used to describe specific embodiments rather than to limit the present invention. Terms without numbers in front are not intended to limit the quantity but to represent the existence of at least one article cited herein. Terms “comprising”, “having”, “including”, and “containing” should be interpreted openly (i.e. “including but not limited to”).

The mention of a numerical range replaces mention of individual numbers within the range, and unless cited otherwise, each number is applied to the specification as if individually mentioned in the specification. The end values of all of the ranges are included in the range and can be individually combined.

All methods mentioned in the specification may be performed in a suitable order unless noted otherwise or explicitly contradicted within the context. The use of any one embodiment and all embodiment, or exemplary language (e.g., “such as” or “like to”), unless included in the claims, is used to more clearly describe the present invention rather than to limit the scope of the present invention. Any language herein outside of the claims should not be interpreted as a necessity of the present invention. Unless defined otherwise, technical and scientific terms used herein each has a meaning ordinarily understood by those of ordinary skill in the art to which the present invention belongs.

The exemplary embodiments of the present invention include the best mode known to the inventors to perform the present invention. Variations in the exemplary embodiments can become clear to those skilled in the art when reading the descriptions above. It is expected that the inventors suitably use such variations, and embody the present invention by different methods described in the specification. Thus, the present invention, as allowed by the patent law, includes equivalents and all modifications of the gist of the present invention mentioned in the accompanying claims. Moreover, all possible variations with any combination of the above-mentioned components are included in the present invention, unless explicitly stated otherwise or contradicting the context. Although the present invention is described and shown by exemplary embodiments, those skilled in the art will understand well that there can be various changes in the form and details without departing from the spirit of the invention and range defined by the claims below.

Hereinafter, the configuration and effects of the present invention will be described in further detail with reference to examples and experimental examples. However, the following examples and experimental examples are merely provided to illustrate the present invention to help understanding the present invention, and the scope of the present invention is not limited thereto.

### Example 1

### Synthesis of Peptides

### 1. Synthesis of Peptides

A peptide of SEQ ID NO: 1 (hereinafter, referred to as “PEP 1”) was prepared according to a conventionally known method of solid phase peptide synthesis. Specifically, peptides were synthesized by coupling each amino acid to another from the C-terminus through Fmoc solid phase peptide synthesis (SPPS) using ASP48S (Peptron, Inc., Daejeon, Korea). Peptides in which the first amino acid at the C-terminus is attached to a resin were used as follows:

NH₂-Lys(Boc)-2-chloro-trityl resin

NH₂-Ala-2-chloro-trityl resin

NH₂-Arg(Pbf)-2-chloro-trityl resin

In all amino acid ingredients used in the synthesis of the peptides, the N-terminus was protected with Fmoc, and the residues were protected with Trt, Boc, t-butylester (t-Bu), and 2,2,4,6,7-pentamethyl dihydro-benzofuran-5-sulfonyl (Pbf) that can be removed in an acid. Examples of the amino acids are as follows:
Fmoc-Ala-OH, Fmoc-Arg(Pbf)-OH, Fmoc-Glu(OtBu)-OH, Fmoc-Pro-OH, Fmoc-Leu-OH, Fmoc-Ile-OH, Fmoc-Phe-OH, Fmoc-Ser(tBu)-OH, Fmoc-Thr(tBu)-OH, Fmoc-Lys(Boc)-OH, Fmoc-Gln(Trt)-OH, Fmoc-Trp(Boc)-OH, Fmoc-Met-OH, Fmoc-Asn(Trt)-OH, Fmoc-Tyr(tBu)-OH, Fmoc-Ahx-OH, Trt-Mercaptoacetic acid.

As a coupling reagent, 2-(1H-Benzotriazole-1-yl)-1,1,3,3-tetamethylaminium hexafluorophosphate (HBTU)/N-Hydroxxybenzotriazole (HOBt)/4-Methylmorpholine (NMM) was used. Fmoc deprotection was carried out using piperidine in 20% DMF. To separate the synthesized peptide from the resin and remove the protective group of the residue, a cleavage cocktail [trifluoroacetic acid (TFA)/triisopropylsilane (TIS)/ethanedithiol (EDT)/H₂O=92.5/2.5/2.5/2.5] was used.

Each peptide was synthesized by a repeated process of reacting each of corresponding amino acids to the starting amino acid protected by the amino acid protective group while bound to a solid phase scaffold, washing the resulting product with a solvent, and performing deprotection. After being detached from the resin, the synthesized peptide was purified by HPLC, coupling validated by mass spectrometry (MS), and lyophilized.

The purity of all peptides used in the embodiment was 95% or higher as measured by high-performance liquid chromatography.

A specific process for preparing the peptide PEP1 according to the present invention will be described as follows.

1) Coupling

The amino acid (8 equivalents) protected with NH₂-Lys(Boc)-2-chloro-trityl resin, and a coupling reagent [HBTU (8 equivalents)/HOBt (8 equivalents)/NMM (16 equivalents)] dissolved in DMF were mixed together and incubated at room temperature for 2 hours. The resulting product was sequentially washed with DMF, MeOH, and DMF.

2) Fmoc deprotection

Piperidine in 20% DMF was added to the resulting product, and the mixture was incubated twice at room temperature for 5 minutes and then sequentially washed with DMF, MeOH and DMF.

3) The peptide backbone [NH₂-E(OtBu)-A-R(Pbf)-P-A-L-L-T(tBu)-S(tBu)-R(Pbf)L-R(Pbf)-F-I-P-K(Boc)-2-chloro-Trityl Resin] was prepared by repeating reactions 1 and 2.

4) Cleavage: The peptide was separated from the completely-synthesized resin by adding a cleavage cocktail to the resin.

5) After adding cooling diethyl ether to the obtained mixture, a peptide obtained by centrifugation was precipitated.

6) Following purification by Prep-HPLC, a resulting product was analyzed by LC/MS to identify molecular weight, and lyophilized to prepare a powder.

### Example 2

### Preparation of Dendritic Cells Activated by Antigens (Peptides)

A method for preparing dendritic cells comprises a process of preparing mononuclear cells by proliferating mononuclear cells from obtained blood, and a process of differentiating the mononuclear cells into dendritic cells. According to the method for differentiating mononuclear cells into dendritic cells, mononuclear cells may be cultured in a medium containing interleukin-4 (IL-4) and may be differentiated into immature dendritic cells. The obtained immature dendritic cells may be cultured in a medium containing tumor necrosis factors-α (TNF-α) and may be differentiated into mature dendritic cells.

1. Separation of Human Peripheral Blood Mononuclear Cells (hPBMCs)

5 to 100 cc of peripheral blood was extracted from a healthy applicant using an evacuated blood collection tube containing an anticoagulant such as heparin. The extracted blood was mixed with phosphate buffered saline (PBS) in a predetermined ratio to dilute, and hPBMCs were separated from the diluted sample through density gradient centrifugation using Lymphoprep or Ficoll-Plque. The separated PBMCs were washed with PBS twice before being used in the experiment. Meanwhile, cryopreserved PBMCs were thawed at 37° C. in a water bath within 1 minute and washed with a cell culture medium twice, before being used in the experiment.

### 2. Separation of CD14-Positive Cells (CD14+)

High-purity CD14+ cells were separated from the PBMCs in high yield through a plastic adherence method and a CD14+ MACS separation method. For the plastic adherence method, entire mononuclear cells were seeded and cultured in a cell culture container for 1 to 2 hours, and then only cells attached to the bottom were used through treatment with Trypsin-EDTA or physical detachment. For the MACS separation method, pure CD14+ cells were separated through a CD14 column using microbeads purchased from MiltenyiBiotic (Auburn, Calif.) according to a manufacturer's manual.

3. Differentiation into Immature Dendritic Cells (imDCs)

CD14+ cells were seeded in a cell culture container to have a concentration of 0.5 to 2×10⁶ cells/ml. More specifically, CD14+ cells were added to a CellGro DC serum-free medium (CellGenix) containing 500 to 1,500 U/ml of interleukin-4 (IL-4) and 500 to 2,000 U/ml a granulocyte-macrophage colony-stimulating factor (GM-CSF) and cultured under conditions of 37° C. and 5% CO₂ for 5 days, and the medium was replaced with a cell culture medium every 2 to 3 days to supplement cytokine.

### 4. PEP1 Antigen (Peptide) Activation of Immature Dendritic Cells

To induce an immune response, 5 to 100 μg/ml of PEP1 was added to imDCs, and then the cells were cultured in a CellGro DC serum-free medium (CellGenix) containing 500 to 1,500 U/ml of IL-4, 500 to 2,000 U/ml of GM-CSF and 10 μg/ml of Keyhole limpet hemocyanin (KLH) under conditions of 37° C. and 5% CO₂ for 18 to 24 hours, thereby inducing antigen activation.

5. Differentiation into Mature Dendritic Cells (mDCs)

To induce maturation of the imDCs right after the antigen activation, the cells were cultured in a CellGro DC serum-free medium (CellGenix) containing 5 to 20 ng/ml of tumor necrosis factors-α (TNF-α), 10 to 20 ng/ml of interleukin-1β (IL-1β), 1,000 to 2,000 U/ml of interleukin-6 (IL-6), and 0.01 to 10 μg/ml of prostaglandin E₂ (PGE₂) under conditions of 37° C. and 5% CO₂ for 1 hour.

### Example 3

### Analysis of Function of Dendritic Cells

### Example 3-1: Analyses of Endocytosis and Cellular Uptake of imDCs

The imDCs have an excellent ability of recognizing and capturing antigens, compared with the mature dendritic cells. The imDCs differentiated according to an example of the present invention were subjected to flow cytometry and confocal microscopy to analyze whether an antigen PEP1 is transported into the cells.

1. Analysis of Endocytosis of imDCs

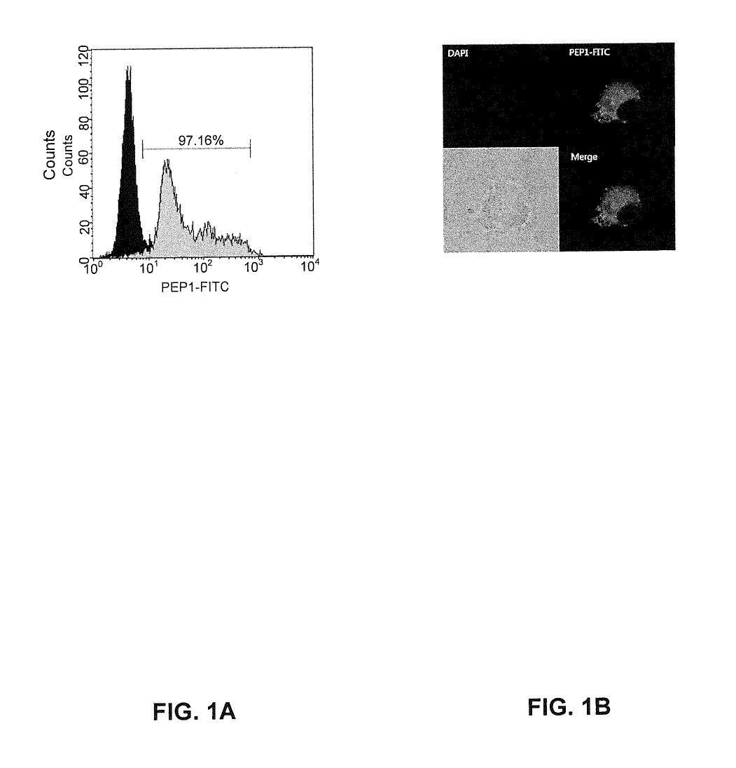

1×10⁶ cells/ml of imDCs were treated with 50 to 100 μg/ml of fluorescein isothiocyanate (PEP1-FITC) and cultured at 37° C. for 30 minutes, 1 hour, or 2 hours. Following the culture, the cells were washed with PBS twice, and endocytosis of imDCs was analyzed using a FACSCalibur (Becton Dickinson). As a control, dendritic cells were cultured at 4° C. for 1 hour. FIG. 1A shows an FACS result denoting that PEP1-treated immature dendritic cells (green right side graph) shifted right, compared with the control (black left side graph). It shows that imDCs recognize PEP1 as an antigen, thereby exhibiting excellent endocytosis performance, and thus serve as antigen-presenting cells. This indicates that initiation of an immune response is stimulated by antigen endocytosis of the immature dendritic cells, presenting the up-taken antigens to MEW molecules through processing, and maturation of the dendritic cells.

2. Analysis of Uptake of imDCs

3×10⁵ cells/ml of immature dendritic cells were seeded in a chamber well, and activated by 50 to 100 μg/ml of PEP1-FITC for 30 minutes, 1 hour, or 2 hours. Following the activation, the chamber well was washed with PBS four times, and the cells were fixed with 2% (v/v) paraformaldehyde at room temperature for 15 minutes. Nuclear staining was carried out with 4′,6-diamidino-2-phenylindole (DAPI) at room temperature for 15 minutes, and visual analysis of uptake of the imDCs was performed through confocal microscopy. FIG. 1B shows a result of analyzing PEP1 antigen uptake of the imDCs, allowing visually identification of the PEP1-FITC penetrated into the imDCs through endocytosis (green).

### Example 3-2: Analysis of T Cell Stimulatory Capacity of PEP1-Activated Dendritic Cells

Dendritic cells activate cytotoxic T lymphocytes (CTLs) to attack target cells to treat a disease. Therefore, the T cell proliferation response and cytokine secretion capacity of PEP1-activated dendritic cells were analyzed.

### 1. Analysis of T Cell Proliferation Response of PEP1-Activated Dendritic Cells

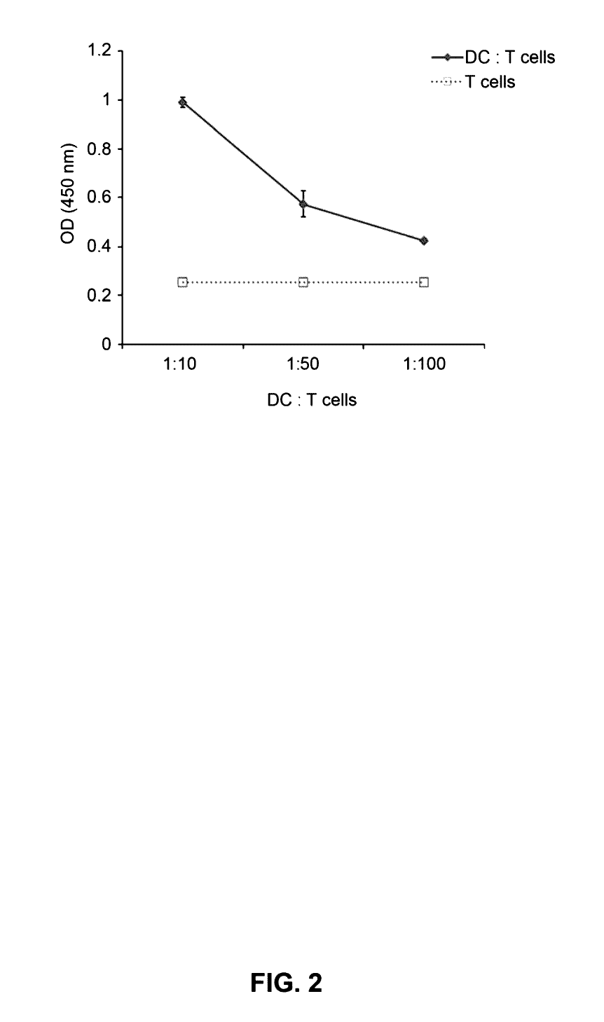

Induction of the T cell proliferation by PEP1-activated dendritic cells was analyzed through mixed lymphocyte reactions (MLRs). The mixed lymphocyte reaction (MLR) was known as a typical method for measuring the activation of a common stimulation molecule, which is a standardization technique for evaluating the function of antigen-presenting cells. The PEP1-activated dendritic cells were mixed with T cells at a ratio of 1:10, 1:50, or 1:100, and cultured in a 96-well plate for 72 hours. Following the culture, a T cell proliferating ability was assessed using a BrdU Cell Proliferation Assay Kit (Cell Signaling Technology, BrdU incorporation). Specifically, a BrdU solution was added to the 96-well plate in which a mixed culture was completed to give a final concentration that is one fold, and cultured at 37° C. for 1 to 24 hours. The plate was centrifuged at 300 g for 10 minutes to remove the BrdU-contained medium, and 100 μl of a fixing/denaturing solution was added to the 96-well plate, followed by a reaction at room temperature for 30 minutes. 100 μl of BrdU detection antibodies were added to the 96-well plate to perform a reaction at room temperature for 1 hour and washed with a washing solution three times. 100 μl of horseradish peroxidase (HRP)-binding secondary antibodies were added to the 96-well plate to perform a reaction at room temperature for 30 minutes and washed with a washing solution three times. Finally, 100 μl of a coloring reagent (tetramethylbenzidine, TMB) was added to perform a reaction at room temperature for 30 minutes, and 100 μl of a coloring stop reagent was added, followed by analyzing the antibodies at 450 nm using an ELISA reader. Many cell aggregates were formed when the dendritic cells were cultured with T lymphocytes, and these aggregates are a group of T lymphocytes stimulated by dendritic cells. FIG. 2 shows the stimulating ability of dendritic cells, indicating that, as the concentration of the PEP1-activated dendritic cells was increased to 1:100, 1:50 or 1:10, optical density (OD) of the dendritic cells, indicating the ability of stimulating T cell proliferation, was increased to 0.42, 0.57 or 0.98, respectively. This shows that the PEP1-activated dendritic cells serve as antigen-presenting cells to induce an immune response to T cells through a crosslinking reaction with the T cells.

### 2. Analysis of Cytokine Secretion Capacity of PEP1-Specific T Cells

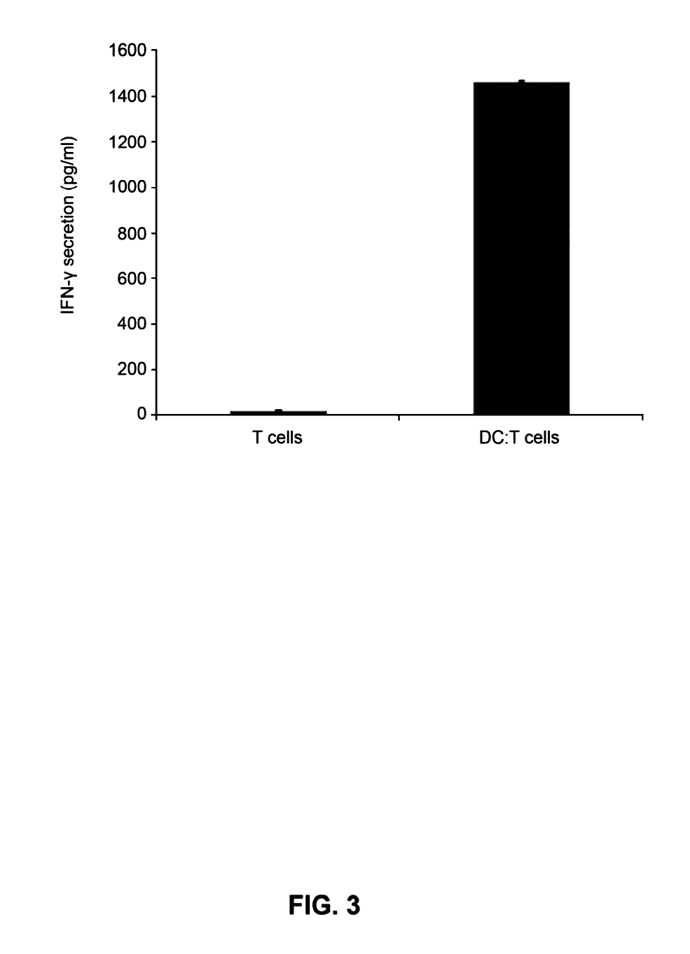

PEP1-activated dendritic cells were mixed with T cells and cultured for 72 hours, and then a culture solution was harvested. The culture solution was centrifuged at 1,600 rpm and 4° C. for 5 minutes to obtain a supernatant, and then a cytokine secretion capacity was analyzed using an IFN-γ ELISA kit (R&D Systems) according to a manufacturer's manual. Specifically, 100 μl of a diluent was added to a 96-well plate to which polyclonal antibodies IFN-γ were attached, and reacted at room temperature for 2 hours. The plate was washed with a washing solution four times, 200 μl of HRP-added IFN-γ secondary antibodies (IFN-γ Conjugated horseradish peroxidase) were added thereto and reacted at room temperature for 2 hours. The resulting plate was washed with a washing solution four times, stained with 200 μl of a coloring reagent at room temperature for 30 minutes, and reacted with 50 μl of a coloring stop reagent, followed by an analysis at 450 nm using an ELISA reader. FIG. 3 shows levels of IFN-γ secreted from PEP1-specific T cells, indicating that the level of IFN-γ secretion was 1,470.2±4.3 pg/ml in the PEP1-specific T cells, which was approximately 59.3-fold higher than that of the control (T cells), which was 24.2±0.8 pg/ml.

### Example 4

### Analysis of Immune Response Effect of Dendritic Cells Activated by Peptides Including PEP1 in Clinical Step

As described above, dendritic cells activate CTL to attack target cells to treat a disease. Accordingly, dendritic cells activated by peptides including PEP1 were prepared, administered to each individual, and subjected to an ELISPOT assay to observe an increase in T cell responses to each peptide in each individual.

### 1. Experimental Subjects and Methods

The experiments were performed on four patients with different diseases (cancers), and a dendritic cell immunotherapy method is as follows. Mononuclear cells were collected from peripheral blood extracted from each patient and cultured. Immature dendritic cells differentiated in the mononuclear cell culture were subjected to antigen activation with tumor-associated antigen peptides including PEP1. When the dendritic cells became mature, antigen activation was carried out once more with the same peptides. The activated mature dendritic cells were separated and then administered to each patient.

More specifically, dendritic cells and NK cells used in treatment were prepared by proliferating mononuclear cells obtained from 25 ml of whole blood taken from a patient every two weeks, and differentiating the proliferated mononuclear cells into dendritic cells. That is, in this example, CD14+ mononuclear cells obtained from a peripheral blood vessel of the patient were cultured with IL-4 and GM-CFS for 6 days and proliferated, and then treated with a Streptococcus agent OK-432 for maturation. In addition, in the preparation of the dendritic cells, the dendritic cells were activated by WT1, MUC1 and other antigens.

According to cancer characteristics of a patient undergoing a gene test, an antigen test and a tumor marker test, a substance having a similar function to a tumor-associated antigen (a cancer cell lysate, tumor-specific protein, or tumor-associated peptide) was added to the dendritic cells to enhance a cancer-recognizing ability of the dendritic cells, which is an activation process. For dendritic cell activation, as tumor-associated antigens that can be used in the present invention but not limited thereto, WT1, MUC-1, CA125, MAGE-A3, CEA, NY-ESO1, survinin, Her2, and the telomerase-derived peptide PEP1 were used. As an activation method but not limited to a specific example, the dendritic cells may be cultured with such a tumor-associated antigen. The activation may be performed on immature dendritic cells or mature dendritic cells. In one specific example of the present invention, mononuclear cells were collected from a patient's peripheral blood and cultured, immature dendritic cells differentiated from the mononuclear cells were treated with a peptide to activate and thus differentiated into mature dendritic cells, and then the mature dendritic cells were treated with the same peptide for activation. An amount of the antigen used in the activation may be the same as generally used in the art as follows but is not limited thereto. For example, PEP1 may be used in an amount of 1 to 1000 μg/ml along with the antigens, and 20 μg/ml of WT1, 20 μg/ml of MUC1, 20 μg/ml of CEA, 500 μg/ml of CA125, and 20 μg/ml of HER2 may be used. The dendritic cells (1×10⁷) prepared as described above were intradermally injected six times every 14 days.

The following experiment was performed to examine whether dendritic cell immunotherapy leads to an increase in T cell responses to peptides activated by an antigen. A buffy coat layer was separated from the peripheral blood of a patient subjected to the dendritic cell immunotherapy using a ficoll method (centrifugation using a ficoll), and mononuclear cells were obtained therefrom. Following 3 to 4-day culture, an ELISPOT assay was carried out. The ELISPOT assay was performed according to a protocol of an ImmunoSpot kit (Celluluar Technology Limited, USA). Specifically, T cells were dispensed at a concentration of 1×10⁵ cells per well in an ImmunoSpot plate. As peptide stimulants (antigen peptides), Peptivator WT1, Peptivator NY-ESO1 (Miltenyi Biotec., Germany), MUC-1, MAGE-A3, Survivin and PEP1 were added to each well at an optimum concentration and cultured for 48 hours. IFN-γ released from a helper T cell 1 (Th1) response and IL-4 released from a helper T cell 2 (Th2) response, which were involved in CTL activation, were stained with specific antibody-detecting reagents. Cells releasing IFN-γ were stained red, and cells releasing IL-4 were stained blue. After staining, the plate was examined using an immunoSpot reader (Cellular Technology Ltd., USA) to quantify spots.

### 2. ELISPOT Results and Analyses for Respective Experimental Subjects

ELISPOT results for respective patients before/after the dendritic cell immunotherapy are as follows:

1) 74-Year-Old Female Patient with Stomach Cancer (Patient 1)

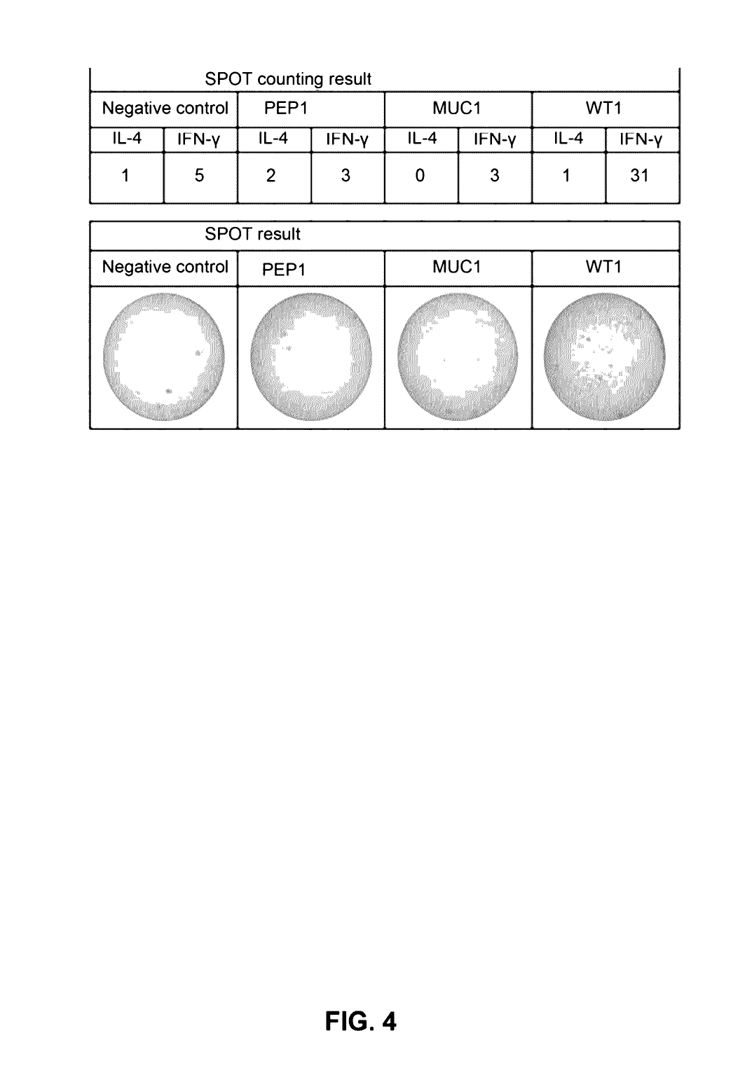

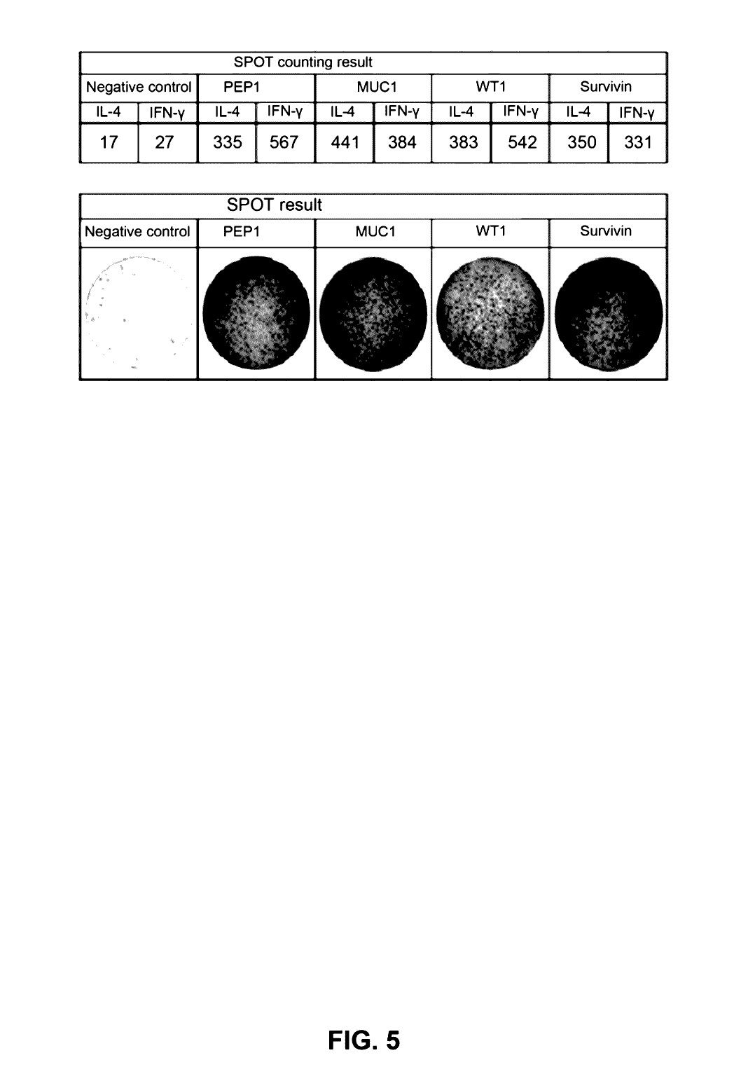

As a result of ELISPOT performed before dendritic cell immunotherapy, compared with a negative control treated with no peptide, in all groups, there was no difference between visual data and numerical data (refer to FIG. 4). A dendritic cell vaccine was administered six times, and a CTL activity induced by stimulation with various peptides was assessed by ELISPOT. As a result of ELISPOT performed after the treatment, spots were increased in all of PEP1, MUC1, WT1 and Survivin groups, compared with the negative control (refer to FIG. 5).

TABLE 2
ELISPOT result (Before dendritic cell vaccine administration)
Number of spots according to ELISPOT assay

| Negative | | | | | | | |
|---|---|---|---|---|---|---|---|
| control | | PEP1 | | MUC1 | | WT1 | |
| IL-4 | IFN-γ | IL-4 | IFN-γ | IL-4 | IFN-γ | IL-4 | IFN-γ |
| 1 | 5 | 2 | 3 | 0 | 3 | 1 | 31 |

TABLE 3
ELISPOT result
(7 days after six times of dendritic cell vaccine administration)
Number of spots according to ELISPOT assay

| Negative | | | | | | | | | |
|---|---|---|---|---|---|---|---|---|---|
| control | | PEP1 | | MUC1 | | WT1 | | Survivin | |
| IL-4 | IFN-γ | IL-4 | IFN-γ | IL-4 | IFN-γ | IL-4 | IFN-γ | IL-4 | IFN-γ |
| 17 | 27 | 335 | 567 | 441 | 384 | 383 | 542 | 350 | 331 |

2) 77-Year-Old Male Patient with Lung Cancer (Patient 2)

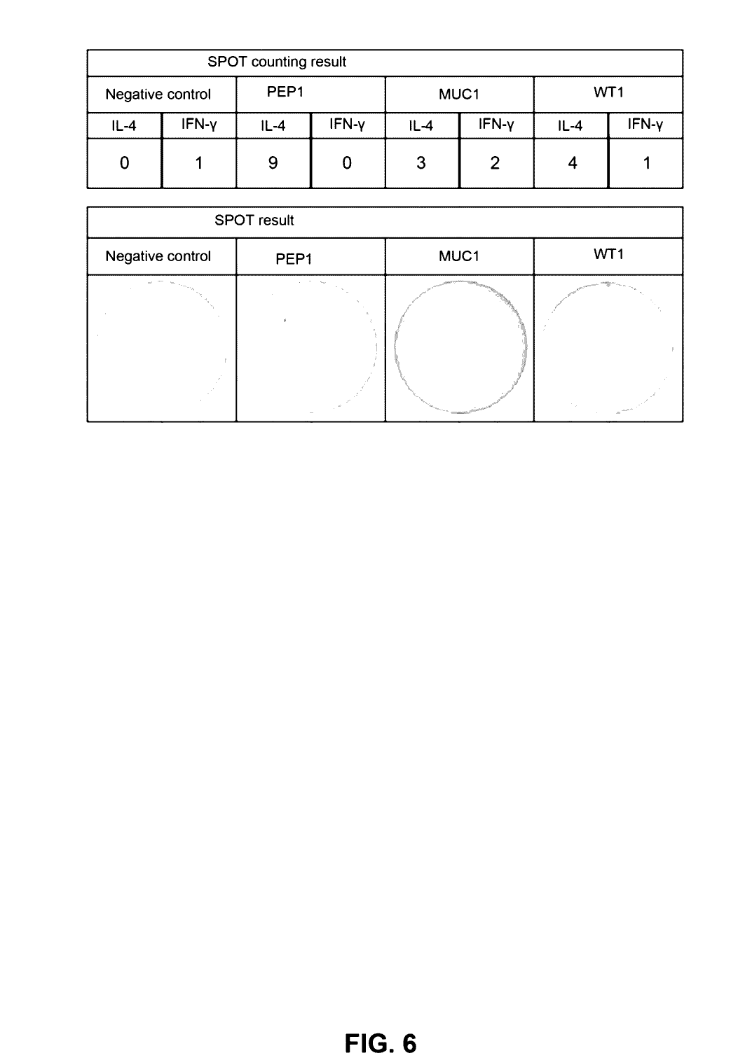

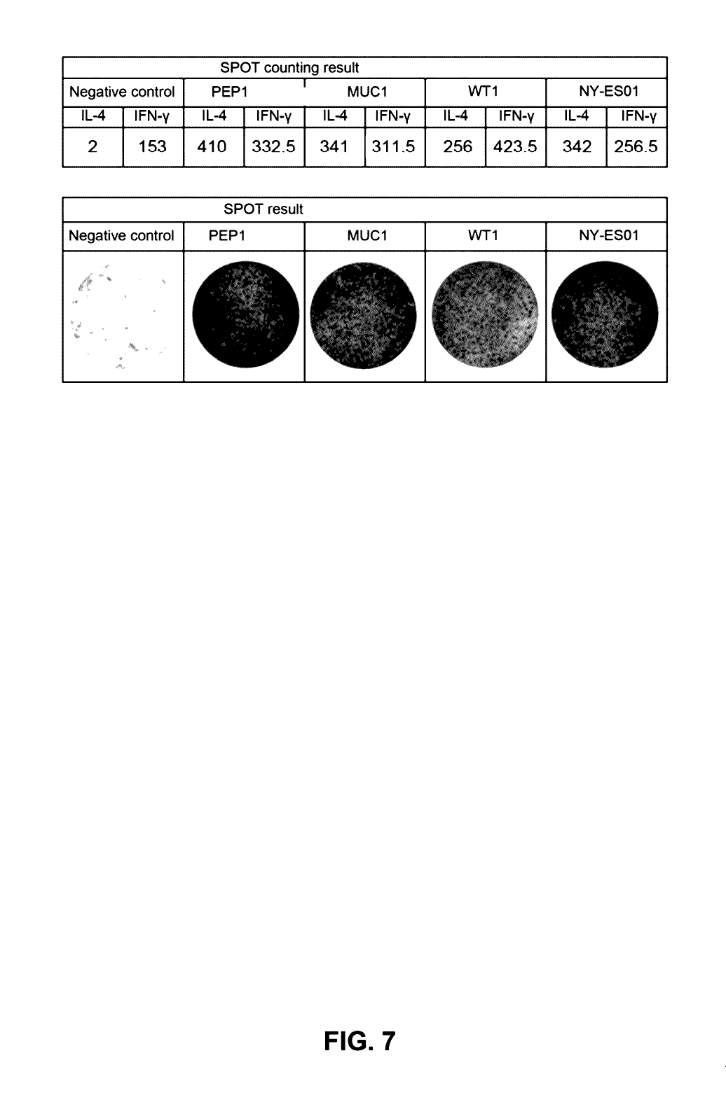

As a result of ELISPOT performed before dendritic cell immunotherapy, compared with a negative control treated with no peptide, in all groups, there was no difference between visual data and numerical data (refer to FIG. 6). A dendritic cell vaccine was administered 6 times, and a CTL activity induced by stimulation with various peptides was assessed by ELISPOT. As a result of ELISPOT performed after the treatment, compared with the negative control, in all of the PEP1, MUC1, WT1, NY-ESO1 groups, spots were increased (refer to FIG. 7). Spots (red) for IFN-γ secretion, indicating activation of an MHC-class-I pathway were detected after 44 days.

TABLE 4
ELISPOT result (Before dendritic cell vaccine administration)
Number of spots according to ELISPOT assay

| Negative | | | | | | | |
|---|---|---|---|---|---|---|---|
| control | | PEP1 | | MUC1 | | WT1 | |
| IL-4 | IFN-γ | IL-4 | IFN-γ | IL-4 | IFN-γ | IL-4 | IFN-γ |
| 0 | 1 | 9 | 0 | 3 | 2 | 4 | 1 |

TABLE 5
ELISPOT result (44 days after six times of dendritic cell vaccine administration)
Number of spots according to ELISPOT assay

| Negative | | | | | | | | | |
|---|---|---|---|---|---|---|---|---|---|
| control | | PEP1 | | MUC1 | | WT1 | | NY-ESO1 | |
| IL-4 | IFN-γ | IL-4 | IFN-γ | IL-4 | IFN-γ | IL-4 | IFN-γ | IL-4 | IFN-γ |
| 2 | 153 | 410 | 332.5* | 341 | 311.5* | 256 | 423.5* | 342 | 256.5* |

Cf) *Due to a large number of IFN-γ spots, an exact number was not obtained.

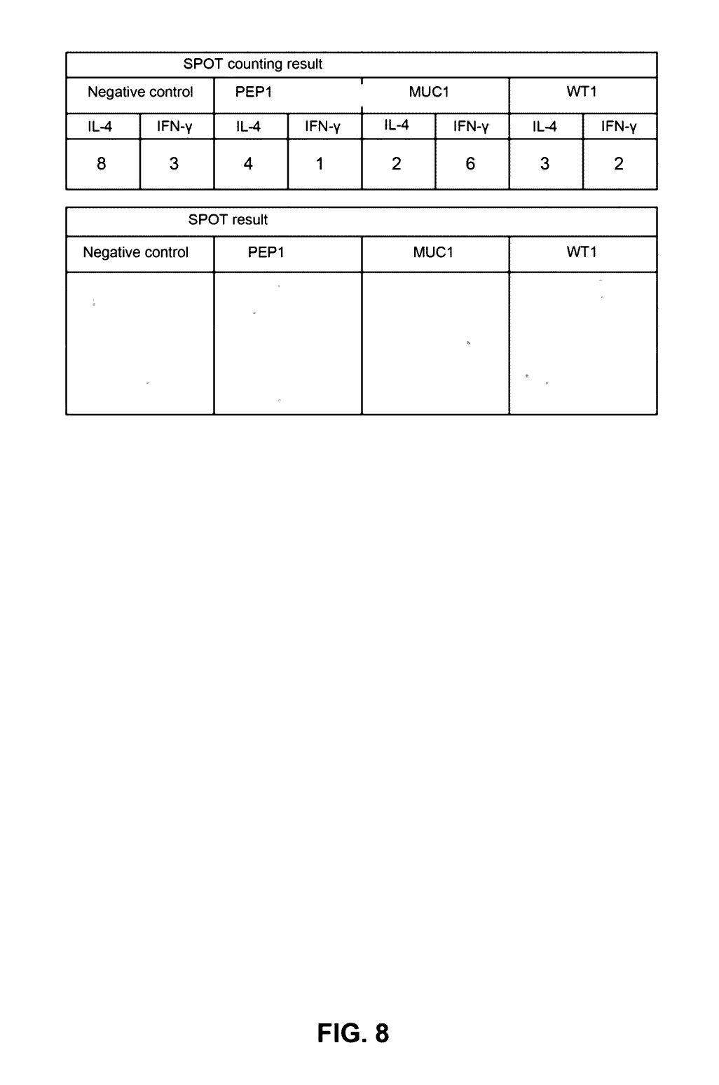

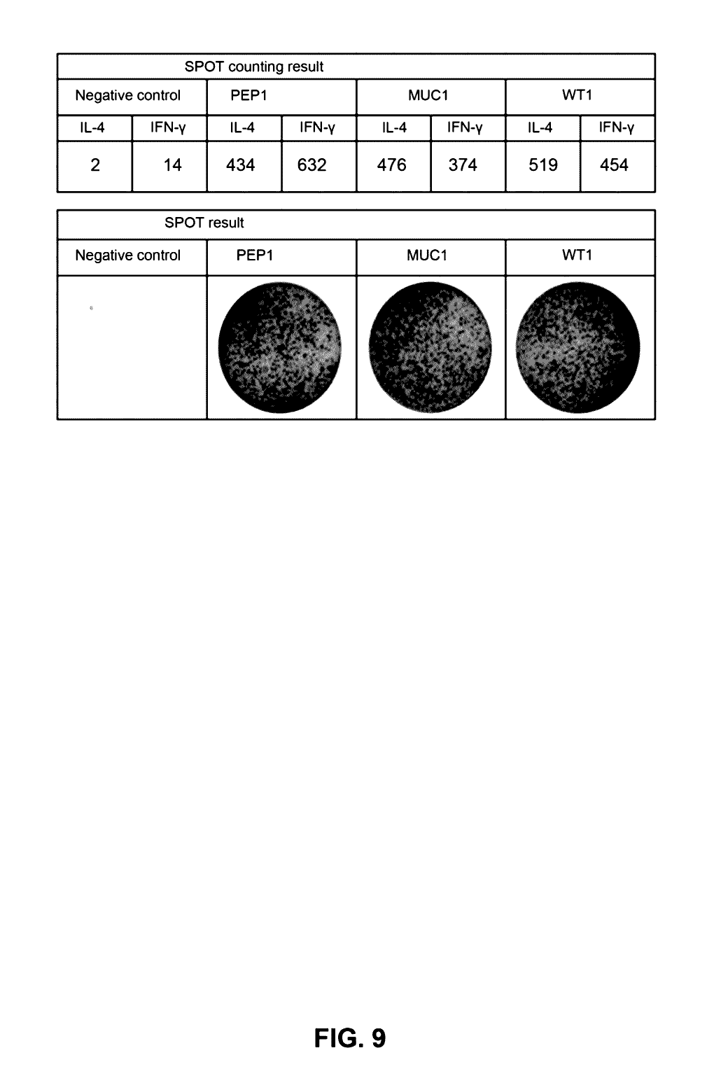

As a result of ELISPOT performed before dendritic cell immunotherapy, compared with a negative control treated with no peptide, in all groups, there was no difference between visual data and numerical data (refer to FIG. 8). A dendritic cell vaccine was administered 6 times, and a CTL activity induced by stimulation with various peptides was assessed by ELISPOT. As a result of ELISPOT performed after the treatment, compared with the negative control, in all of the PEP1, MUC1 the WT1 groups, spots were increased (refer to FIG. 9). IFN-γ spots (red) indicating activation of an MHC-class-I pathway induced by PEP1, MUC1, and WT1 peptides were detected, and IL-4 spots (blue) indicting activation of a MHC-class-II pathway were also detected.

TABLE 6
ELISPOT result (Before dendritic cell vaccine administration)
Number of spots according to ELISPOT assay

| Negative | | | | | | | |
|---|---|---|---|---|---|---|---|
| control | | PEP1 | | MUC1 | | WT1 | |
| IL-4 | IFN-γ | IL-4 | IFN-γ | IL-4 | IFN-γ | IL-4 | IFN-γ |
| 8 | 3 | 4 | 1 | 2 | 6 | 3 | 2 |

TABLE 7
ELISPOT result
(11 days after six times of dendritic cell vaccine administration)
Number of spots according to ELISPOT assay

| Negative | | | | | | | |
|---|---|---|---|---|---|---|---|
| control | | PEP1 | | MUC1 | | WT1 | |
| IL-4 | IFN-γ | IL-4 | IFN-γ | IL-4 | IFN-γ | IL-4 | IFN-γ |
| 2 | 14 | 434 | 632 | 476 | 374 | 519 | 454 |

4) 66-Year-Old Female Patient with Breast Cancer (Patient 4)

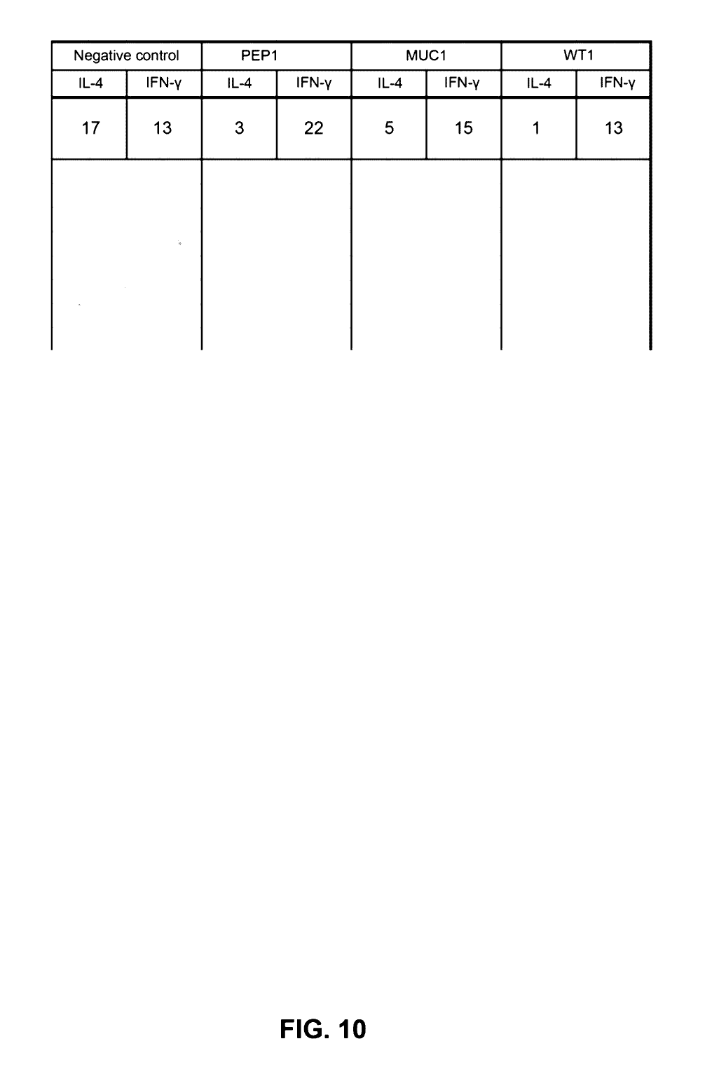

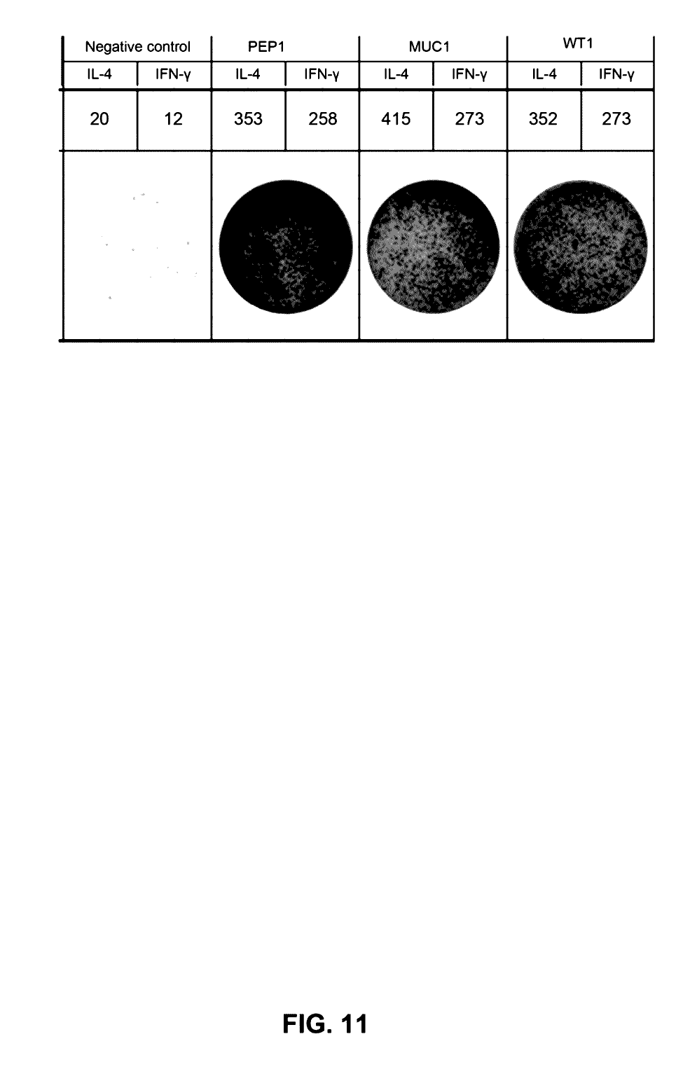

This experiment was performed using a number of T cell-treated dendritic cells that is double the conventional number of cells (2×10⁵ cells/well). As a result of ELISPOT performed before dendritic cell immunotherapy, compared with a negative control treated with no peptide, in all groups, there was no difference between visual data and numerical data (refer to FIG. 10). A dendritic cell vaccine was administered 6 times, and a CTL activity induced by stimulation with various peptides was assessed by ELISPOT. As a result of ELISPOT performed after the treatment, compared with the negative control, in all of the PEP1, MUC1 the WT1 groups, spots were increased (refer to FIG. 11). IFN-γ spots (red) indicating activation of an MHC-class-I pathway were detected after 35 days, and many IL-4 spots (blue) indicting activation of a MHC-class-II pathway were also detected.

TABLE 8
ELISPOT result (Before dendritic cell vaccine administration)
Number of spots according to ELISPOT assay

| Negative | | | | | | | |
|---|---|---|---|---|---|---|---|
| control | | PEP1 | | MUC1 | | WT1 | |
| IL-4 | IFN-γ | IL-4 | IFN-γ | IL-4 | IFN-γ | IL-4 | IFN-γ |
| 17 | 13 | 3 | 22 | 5 | 15 | 1 | 13 |

TABLE 9
ELISPOT result
(35 days after six times of dendritic cell vaccine administration)
Number of spots according to ELISPOT assay

| Negative | | | | | | | |
|---|---|---|---|---|---|---|---|
| control | | PEP1 | | MUC1 | | WT1 | |
| IL-4 | IFN-γ | IL-4 | IFN-γ | IL-4 | IFN-γ | IL-4 | IFN-γ |
| 20 | 12 | 353 | 258 | 415 | 273 | 352 | 273 |

According to the ELISPOT assays performed on the above-mentioned four patients, dendritic cell immunotherapy, which comprises activating dendritic cells by antigen peptides, can detect many spots indicating CTL activation specific for the antigen peptides. This shows that the dendritic cell immunotherapy according to the present invention induces antigen-specific T cell responses.

In addition, it has been known that IFN-γ is secreted from Th1 cells which serve as an MHC-class-1 pathway and activate CTL due to stimulation with various peptides, and IL-4 is secreted from Th2 cells serving as an MHC-class-2 pathway. According to the experiment, it can be deduced that the peptide antigens used in the experiment is involved in activation of the MHC-class-1 pathway and activation of the MHC-class-2 pathway.

### Example 4

### Immunotherapy Using Dendritic Cells Activated by Peptides Including PEP1 and Analysis of Effect of Co-Administration with PEP1 in Clinical Step

An experiment was performed on individuals with cancer which is most suitable for a target treatment. The experiment was performed on the assumption that a dendritic cell therapeutic agent using dendritic cells activated by peptides including PEP1, and a combination of an NK cell therapeutic agent and a therapeutic method for directly administering a PEP-containing immunotherapeutic agent may become an effective therapeutic method to a patient with progressing cancer, without a serious side effect.

The experiment was performed on four persons in consideration of the state of each patient as follows:

1) 89-Year-Old Female with Stage I Lung Cancer (Patient 5)

This patient got a radioablation therapy once in 2012, and got the same therapy once again in 2013 after local recurrence. Also, the patient got primary dendritic cell immunotherapy in 2013. As a result of examining the lung through PET-CT on May, 2014, several masses were found, and levels of the several tumor markers were increased.

Afterward, the patient got immunotherapy using a dendritic cell therapeutic agent and an NK cell therapeutic agent six times each every two weeks. The dendritic cells and the NK cells used in the treatment were obtained from 25 ml of whole blood of the patient extracted every two weeks according to a method disclosed in U.S. Pat. No. 5,577,472 (The Life Science Institute Co. Ltd, Tokyo, Japan). Four peptides (WT-1, MUC-1, PEP1 and MAGE-A3) were used as antigen peptides to activate the dendritic cells. The dendritic cells were administered by intradermal injection around a lymph node, and the NK cells were administered through drip infusion of vein.

Following the immunotherapy including dendritic cell immunotherapy, changes in mass and tumor markers in the patient's lung are shown in Table 10.

TABLE 10

| | | Before | After | Change |
|---|---|---|---|---|
| Tumor | Normal | immuno- | immuno- | after |
| marker | range | therapy | therapy | treatment |
| CA125 (U/ml) | 0 to 35.1 | 38.7 | 15.8 | Reduced |
| NSE (ng/ml) | 0 to 16.3 | 21.1 | 16.7 | Reduced |

As shown in Table 10, mass and tumor markers were apparently reduced, and there were no side effects during the immunotherapy.

2) 47-Year-Old Male with Stage III Pancreatic Cancer (Patient 6)

This patient was diagnosed with pancreatic cancer in 2014, and got oral chemotherapy after partial pancreatectomy in the same year. Right after the patient was selected as an experimental subject, liver metastases were found at two lesions through CT. Also, levels of several tumor markers were increased.

Afterward, the patient had dendritic cell (DC) immunotherapy in combination with NK cell immunotherapy six times every two weeks. In addition, an immunotherapeutic agent including a PEP1 was administered 9 times every two weeks. The immunotherapeutic agent including the PEP1 was administered at a content of 0.56 mg every time. The dendritic cells and NK cells used in the treatment were obtained from 25 ml of whole blood of a patient taken every two weeks. For activation of the dendritic cells, four types of peptides (WT-1, MUC-1, PEP1 and Survivin) served as antigen peptides and pulsed onto the dendritic cells. The dendritic cells (DCs) and PEP1 were administered by intradermal injection around a lymph node, and the NK cells were administered through drip infusion of vein. Also, the patient had FOLFORINOX chemotherapy every two weeks during the immunotherapy.

Liver metastases and changes in tumor markers after immunotherapy including administration of the dendritic cell therapeutic agent activated by the peptides including PEP1 and the immunotherapeutic agent including PEP1 are shown in Table 11.

TABLE 11

| | | Before | After | Changes |
|---|---|---|---|---|
| Tumor | Normal | immuno- | immuno- | after |
| marker | range | therapy | therapy | treatment |
| Dupan2 (U/ml) | 0 to 150 | 323 | 83 | Reduced |
| CA19-9 (U/ml) | 0 to 37 | 649 | 143.3 | Reduced |

As shown in Table 11, cancer metastases and tumor markers were apparently reduced, and there were no side effects during the immunotherapy.

3) 70-Year-Old Female with Stage III Lung Cancer (Patient 7)

This patient was diagnosed with lung cancer in 2011, and had a surgery in the same year, and several chemotherapies in 2013. Right after the patient was selected as an experimental subject, one mass was found in the right lung through CT, and level of one tumor marker was increased.

The patient had dendritic cell immunotherapy and NK cell immunotherapy twice in a row. Since one immunotherapy was performed six times every two weeks, the patient had the immunotherapy 12 times. Also, the immunotherapeutic agent including the PEP1 was administered 11 times every two weeks. The immunotherapeutic agent including the PEP1 was administered at an amount of 0.56 mg every time. The dendritic cells and NK cells used in the treatment were obtained from 25 ml of whole blood of a patient taken every two weeks.

For activation of the dendritic cells, in the first treatment including one to six-time peptide administrations, three types of peptides (WT-1, MUC-1 and Survivin) served as antigen peptides and were pulsed onto the dendritic cells. In the second treatment including 7 to 12-time peptide administrations, five types of peptides (WT-1, MUC-1, NY-ESO1, PEP1 and MAGE-A3) served as antigen peptides and were pulsed onto the dendritic cells. The dendritic cells (DCs) and PEP1 were administered by intradermal injection around a lymph node, and the NK cells were administered through drip infusion of vein. Also, the patient had several chemotherapies during the immunotherapy.

Following the two times of immunotherapy including the dendritic cells (DC) immunotherapy and the PEP1 administration, CT showed that there was almost no change in size of the mass in the right lung of the patient. However, some tumor markers of the patient were reduced, and specific values thereof are shown in Table 12.

TABLE 12

| | | Before | After | Changes |
|---|---|---|---|---|
| Tumor | Normal | immuno- | immuno- | after |
| marker | range | therapy | therapy | treatment |
| NSE (ng/ml) | 0 to 16.3 | 18.1 | 14.9 | Reduced |

As shown in Table 12, cancer metastases and the tumor markers were reduced, and there were no side effects during the immunotherapy.

4) 77-Year-Old Male with Stage III Lung Cancer (Patient 8)

This patient was diagnosed with lung cancer in 2014, and selected as a subject in the same year without having any treatment. Right after the patient was selected as an experimental subject, one mass was found in the left lung and metastases on several lymph nodes were found through CT. Also, levels of tumor markers were increased.

The patient had an immunotherapy using a dendritic cell (DC) therapeutic agent and an NK cell therapeutic agent six times every two weeks. A PEP1 was administered 13 times every two weeks. The immunotherapeutic agent including the PEP1 was administered at an amount of 0.56 mg every time. The immunotherapeutic agent including the PEP1 was administered at an amount of 0.56 mg every time. For activation of the dendritic cells, 6 types of peptides (WT-1, MUC-1, PEP1, CEA, NY-ESO1 and MAGE-A3) served as antigen peptides and were pulsed onto the dendritic cells. The dendritic cells and the PEP1 were administered by intradermal injection around a lymph node, and the NK cells were administered through drip infusion through vein. During the immunotherapy, the patient had 33 radiation treatments.

The immunotherapy including the dendritic cell immunotherapy and PEP1 administration did not show a change in lymph node metastatic state of the patient, inflammation caused by radiation treatment, or an increase of an amount of pleural effusion in the pleural cavity. However, levels of the six types of tumor markers of the patient were reduced, and change in the levels of the total tumor markers are shown in Table 13.

TABLE 13

| | | Before | After | Changes |
|---|---|---|---|---|
| Tumor | Normal | immuno- | immuno- | after |
| marker | range | therapy | therapy | treatment |
| SLX (U/ml) | 0 to 38 | 41.5 | 28.7 | Reduced |
| ICTP (ng/ml) | 0 to 5.5 | 19.1 | 18.9 | Reduced |
| CEA (ng/ml) | 0 to 5 | 8.7 | 6.8 | Reduced |
| CA15-3 (U/ml) | 0 to 27 | 68 | 45.5 | Reduced |
| CA72-4 (U/ml) | 0 to 8 | 51.5 | 16.4 | Reduced |
| CYFRA (ng/ml) | 0 to 3.5 | 22.8 | 5 | Reduced |
| CA125 (U/ml) | 0 to 35.1 | 15.9 | 92.7 | Increased |
| CA19-9 (U/ml) | 0 to 37 | 11 | 40.2 | Increased |
| SCC (ng/ml) | 0 to 1.5 | 2.6 | 2.9 | Increased |
| proGRP (pg/ml) | 0 to 81 | 113 | 124.4 | Increased |

The results for the four patients show that immunotherapy with dendritic cells activated by the peptides including PEP1 has an anticancer effect exhibited by the reduction of the tumor markers of the patient. Also, compared to Patient 1 only having the dendritic cell immunotherapy and the NK cell treatment, Patient 2 receiving the treatment in combination with the PEP1 administration showed further reduction of the tumor markers. This result may show that the co-administration of PEP1 gives a further improved immunotherapeutic effect and increased immunity due to the PEP1 activation in the dendritic cell immunotherapy.

Additionally, Patient 3 and Patient 4 are patients further having chemotherapy (Patient 3) and radiation therapy (Patient 4) as well as the dendritic cell immunotherapy, and in both of the patients, reductions of the tumor markers were shown. This shows that, since, in general, the chemotherapy and radiation therapy considerably reduce the immune responses, the dendritic cell immunotherapy according to the present invention raises an immune response even in an environment with a reduced immune response, resulting in a reduction of the tumor markers. Also, Patient 3 and Patient 4 are the patients with treatments in combination with PEP1, which may also result in an increased immune response and an immune-boosting effect caused by PEP1 activation of the dendritic cell immunotherapy.

According to the above-described examples, it can be seen that dendritic cell immunotherapy using dendritic cells activated by peptides including PEP1 reinforces an immune response and thus exhibits an excellent disease treatment effect. Also, when both the dendritic cell immunotherapy and the PEP1 administration are used, compared to when only the dendritic cell immunotherapy is used, an excellent effect is shown. Therefore, the dendritic cell immunotherapy according to the present invention may be developed as an effective therapeutic agent for a target-specific disease and thus may provide a suitable treating method to an individual requiring target-specific treatment including cancer treatment. Also, it is expected that combined administration of a dendritic cell immunotherapeutic agent and PEP1 may also show an excellent target-specific disease treating effect.

An immune response-activating composition comprising dendritic cells activated by peptides including a peptide having a sequence of SEQ ID NO: 1 or a peptide having a sequence having 80% homology with the sequence of SEQ ID NO: 1 according to the present invention exhibits effects on reduction of factors causing diseases in an individual having disease or disorder symptoms requiring a target-specific treatment and an increase in immune responses in the individual and thus is expected to provide a method for treating a disease or disorder symptom requiring a target-specific treatment, including a tumor disease.

It will be apparent to those skilled in the art that various modifications can be made to the above-described exemplary embodiments of the present invention without departing from the spirit or scope of the invention. Thus, it is intended that the present invention covers all such modifications provided they come within the scope of the appended claims and their equivalents.

1. An immune response-activating composition for generating an immune response to infected or degenerated cells comprising dendritic cells activated by an isolated peptide of SEQ ID NO: 1.

2. (canceled)

3. The composition of claim 1, wherein the dendritic cells are further activated by at least one antigen-derived peptide selected from the group consisting of WT1, MUC-1, CA125, MAGE-A3, CEA, NY-ESO1, Survinin and Her2.

4. The composition of claim 1, wherein the composition treats inflammation or cancer by generating an immune response to infected or degenerated cells.

5. The composition of claim 4, wherein the cancer is at least one selected from the group of pancreatic cancer, lung cancer, breast cancer, prostatic cancer, liver cancer and renal cancer.

6. The composition of claim 1, wherein the dendritic cells are derived from mononuclear cells cultured after being selected from peripheral blood of an individual requiring administration of the composition.

7. The composition of claim 1, wherein the composition is co-administered with an immunotherapeutic agent comprising a peptide of SEQ ID NO: 1.

8. The composition of claim 1, wherein the composition is co-administered with a natural killer (NK) cell therapeutic agent.

9. The composition of claim 1, wherein the composition is co-administered with at least one anticancer agent for chemotherapy or targeted anticancer agent.

10. The composition of claim 1, wherein the composition is used in combination with radiation therapy.

11. A method for activating an immune response comprising:
administering the immune response-activating composition of claim 1 to an individual having a disease or disorder requiring a target-specific treatment, to generate an immune response.

12. The method of claim 11, wherein the composition is administered by intradermal injection around a lymph node every two weeks.

13. The method of claim 11, wherein the composition is co-administered with an immunotherapeutic agent comprising an amino acid sequence of SEQ ID NO: 1.

14. An immune response-activating kit for generating an immune response to inflammation or cancer, the kit comprising the immune response-activating composition of claim 1 and an instruction directing administration of the immune response-activating composition of claim 1.

15. The kit of claim 14, wherein the kit further comprises an immunotherapeutic agent comprising a peptide of SEQ ID NO: 1 and the instruction further directs administration of the immune response-activating composition comprising the dendritic cells of claim 1 and the immunotherapeutic agent of SEQ ID NO: 1 as pharmaceutically active ingredients through intradermal injection around a lymph node every two weeks.
